(19) United States
(12) Patent Application Publication
(10) Pub. No.: US 2019/0322998 A1
(43) Pub. Date: Oct. 24, 2019

(54) ONE POT ASSEMBLY

(71) Applicant: University of Southampton, Highfield (GB)

(72) Inventor: Ali Tavassoli, Highfield (GB)

(21) Appl. No.: 15/499,841

(22) Filed: Apr. 27, 2017

(51) Int. Cl. C12N 15/10

(52) U.S. Cl. CPC C12N 15/10

(57) ABSTRACT

Disclosed herein are methods of synthesising a double stranded polynucleotide, the methods including providing a set of self-templating oligonucleotides wherein the set of self-templating oligonucleotides encodes a double stranded polynucleotide sequence of interest; annealing the oligonucleotides together so that they form the double stranded polynucleotide sequence with the oligonucleotides in the correct order and adjacent oligonucleotides in the same strand close enough to each other that a covalent bond could form between the 5′ end of one oligonucleotide and the 3′ end of an adjacent oligonucleotide; and covalently bonding the backbones of adjacent oligonucleotides in the same strand to each other so that a covalent bond is formed between the 5′end of an oligonucleotide to the 3′end of the adjacent oligonucleotide to provide the double stranded polynucleotide of interest, wherein the covalent bonds between adjacent oligonucleotides can be read-through accurately by a DNA polymerase or an RNA polymerase.

### TECHNICAL FIELD

Embodiments relate to one pot methods for synthesis of double stranded poly-nucleotides comprising at least a whole open reading frame, and in particular, embodiments relate to covalently joining oligonucleotides to form synthetic genes that are transcribed and translated in vivo.

### BACKGROUND

The chemical synthesis of oligonucleotides and their enzyme-mediated assembly into genes and genomes has significantly advanced multiple scientific disciplines. While current approaches are widely employed, they are not without their shortcomings. First, the reliance on enzymes for assembly is not amenable to automation, increasing the time and effort required. Second, enzymatic assembly does not allow the incorporation of epigenetic information and/or modified bases.

The ability to design and synthesize large fragments of DNA has underpinned and revolutionized multiple fields, including cell biology, biotechnology and synthetic biology. While chemical synthesis of short oligonucleotide fragments (<100 bases) is routine, the synthesis of longer fragments is often plagued by poor yields and high error rates which occur as a function of oligonucleotide length. As a result, large DNA fragments need to be assembled from multiple short oligonucleotides using enzymes. Current assembly approaches typically make use of PCR amplification or enzymatic ligation, and although these approaches are well established and form the cornerstone of current gene and genome synthesis efforts, they have some limitations. First, chemical modifications and epigenetic information cannot be introduced into a gene or genome site-specifically as modified bases are not differentiated by PCR enzymes. Second, current assembly methods do not readily lend themselves to automation, and therefore require significant effort and time. Third, the assembly reactions are often low yielding and carried out at a small scale, so require a final PCR amplification step to isolate the full length product from the partially assembled fragments.

Regardless of these limitations, enzymatic assembly of genes and genomes has been used to prepare genomes of over a million base pairs and is routinely employed on a smaller scale for the preparation of genes in everyday research. Previous studies have attempted to chemically ligate synthesised oligonucleotides to form longer DNA molecules as described in WO2008/120016, Kumar et al. 2007, J Am Chem Soc 129, 6859-6864, Kocalka et al. 2008, Chem Bio Chem, 9, 1280-1285, and El-Sagheer et al. 2009, J Am Chem Soc. 131(11), 3958-3964. The drawback with these molecules was that, because they contained unnatural linkages between the oligonucleotides they were not fully active in a biological system. DNA and RNA polymerases could not read these nucleotide sequences accurately and mis-read or missed out nucleotides when trying to replicate the sequences.

Given the challenge of preparing ever increasing lengths of DNA at larger scale and the need for chemically modified DNA constructs, alternative approaches to current DNA assembly methods are needed.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 shows one pot gene synthesis by click-DNA ligation. (A) Schematic representation of the one-pot click-ligation strategy. (B) CuAAC reaction between adjacent terminal cytidine and thymidine residues giving rise to a 1,4-linked 1,2,3-triazole. (C) Our approach is exemplified with the assembly of a gene encoding iLOV. The sequences of the individual oligonucleotides used for the assembly of the whole gene is shown. The positions of the triazole linkages in the sense and antisense strands are highlighted by the boxes and restriction sites are in italics.

FIG. 2 shows synthesis of alkyne-modified cytidine and loading onto solid support in preparation for oligonucleotide synthesis. The alkyne-modified cytidine was coupled to an amino functionalized CPG resin that had been treated with succinic anhydride prior to coupling.

FIG. 3 shows characterisation of click-ligated iLOV gene. (A) PAGE analysis of the constituent modified oligonucleotides in the absence of Cu^{I} (Lane 1), the crude product of the click reaction (Lane 2), visualized under UV light. The position of the click-iLOV product is indicated. In Lane 2, the lower molecular weight bands have been attributed to cyclized and truncated products. (B) Agarose gel of the purified click-linked iLOV gene (lane 2), which shows a 350 bp band (ladder in lane 1); inserted image shows a vial of the purified click-linked iLOV gene. (C) CD spectra of canonical and click-iLOV genes (D) PCR amplification of click-iLOV gene. Lanes 2 and 4 show control experiments in which the unligated modified oligonucleotides where used as template for amplification by Taq and Pfu DNA polymerases. Lanes 3 and 5 show the results of amplification of the click-linked iLOV gene by Taq and Pfu DNA polymerases. In both lanes, a band was observed between 300-350 bp, corresponding to the 335 bp click-ligated iLOV gene. Lane 1 is a DNA Ladder. (E) Primer extension analysis of the sense strand of click-iLOV gene using Klenow fragment (exo)⁻ DNA polymerase. Lane 1: negative control in which the enzyme was omitted from the reaction. The positions of the template and primer are indicated. Lane 2: primer extension reaction; the primer was depleted and extended to the same length as the template. (F) Quantitative real-time PCR (qPCR) analysis of the effect of triazole linkers on DNA replication. Amplification of fragments of click-linked iLOV gene (green) containing increasing numbers of triazoles and their canonical equivalent (black) were assessed by qPCR. The binding sites of the forward primer have been represented schematically above each graph, with replication through (i) one, (ii) two, (iii) three and (iv) four triazoles assessed. Reverse primer is not shown, but anneals to the 3′-terminus of the upper strand.

FIG. 4 shows assembly of click-ligated iLOV plasmid. The iLOV gene was assembled from functionalized oligonucleotides by click DNA ligation; the gene was designed so that it contained ‘sticky ends’ ready for ligation into a plasmid backbone cleaved by NdeI and EcoRI restriction endonucleases. The pRSET-mCherry plasmid, digested with NdeI and EcoRI to excise the mCherry gene (encoding a red fluorescent protein) was used as the backbone. The click-linked iLOV gene (encoding a green fluorescent protein) was ligated into this backbone using T4 DNA ligase.

FIG. 5 shows biocompatibility of click-linked iLOV in E. coli cells. (A) E. coli were transformed with plasmids containing click-linked iLOV or ligase-assembled iLOV, and incubated on LB-agar plates for 16 h. The top image was captured under white light. The bottom image shows iLOV (green, 488 nm) and mCherry (red, 610 nm) fluorescence on the same plate, with the green and red fluorescence images merged. Colonies containing iLOV or mCherry are readily identified (B) Percentage of colonies displaying the green, white and red phenotypes when cells were transformed with plasmids containing the click-ligated or ligase-assembled iLOV. The total number of colonies assessed is shown (n=10). Plasmids were isolated from 50 colonies displaying green fluorescence for each type of insert, and submitted for DNA sequencing. The percentage of green colonies containing the correct sequence for iLOV is shown beneath the graph. (C) Representative sequence electropheregram from a single colony of KRX E. coli transformed with click-linked iLOV. The click-linked gene has been correctly replicated in the bacteria and is error free. The red boxes highlight the position of the triazole-linked CT dinucleotides in the sense strand, while the yellow boxed highlight triazoles linked CT dinucleotides in the antisense strand.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

All publications referred to in this application are hereby incorporated by reference in their entirety.

Embodiments herein provide purely chemical methods for linking and assembling synthetic oligonucleotides into larger fragments, which methods overcome the limitations of existing enzymatic DNA assembly methods, and which reduce the time and cost associated with gene and genome synthesis. It is an aim of the present disclosure to provide a method of synthesising a double stranded polynucleotide comprising at least a whole open reading frame that can be transcribed and translated in vivo to express a functional protein. In a first aspect the present disclosure provides a method of synthesising a double stranded polynucleotide comprising at least a whole open reading frame, wherein the method comprises:

I) providing a set of self-templating oligonucleotides wherein the set of self-templating oligonucleotides encodes a double stranded polynucleotide that is at least a whole open reading frame;

II) annealing the oligonucleotides together so that they form the double polynucleotide, with the oligonucleotides in the correct order and wherein adjacent oligonucleotides in the same strand are close enough to each other that a covalent bond could form between the 5′ end of one oligonucleotide and the 3′ end of an adjacent oligonucleotide; and

III) covalently bonding the backbones of adjacent oligonucleotides in the same strand to each other so that a covalent bond is formed between the 5′end of an oligonucleotide to the 3′end of the adjacent oligonucleotide to provide the double stranded polynucleotide comprising at least a whole open reading frame, wherein the covalent bonds between adjacent oligonucleotides can be read-through accurately by a DNA polymerase or an RNA polymerase in vivo.

The polynucleotide may be at least a whole open reading frame, for example an open reading frame encoding one or more proteins or protein fragments. The polynucleotide may comprise a whole gene or genome. The polynucleotide may comprise an open reading frame encoding one or more proteins or protein fragments as well as regulatory sequences for correct expression of the protein or protein fragment in a cell. For example, the polynucleotide may encode upstream regulatory sequences, such as promoters and enhancers, start codons, stop codons, downstream regulatory sequences as well as the polypeptide sequence. The polynucleotide sequence, gene or genome may include epigenetically modified base, including methyl-cytosine, or hydroxymethyl-cytosine, or other chemical moieties, such as a fluorescent group, or added chemical functional groups for further decoration of the gene after assembly (for example bromination).

The oligonucleotides may be a set of oligonucleotides that, together as a set, encode the whole sequence of the polynucleotide. The set of oligonucleotides comprises two or more, three or more, five or more or ten or more oligonucleotides that encode the whole sequence of the sense strand of the polynucleotide and two or more, three or more, five or more or ten or more oligonucleotides that encode the whole sequence of the antisense strand of the polynucleotide.

The set of oligonucleotides may comprise more than 2, more than 3, more than 4, more than 5, more than 6, more than 7, more than 8, more than 9, more than 10, more than 15 or more than 20, more than 50, more than 100 or more than 200 oligonucleotides. The set of oligonucleotides may comprise the same number of oligonucleotides encoding the sense strand and the antisense strand.

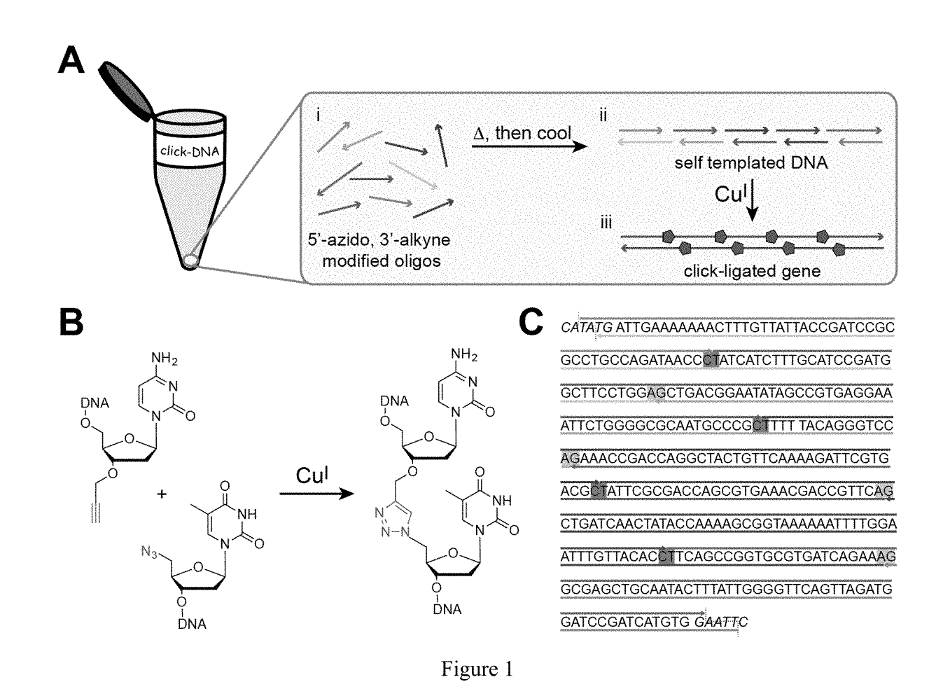

The set of oligonucleotides may be self-templating. Self-templating means that, each oligonucleotide sequence overlaps with at least part of the sequences of two or more adjacent oligonucleotides from the opposite strand. Each oligonucleotide anneals to two or more adjacent oligonucleotides from the opposite strand so that each oligonucleotide acts as a template to ensure that some of the oligonucleotides on the opposite strand are in the correct order and correctly positioned. The set of oligonucleotides may be designed so that the oligonucleotides on each strand assemble in the correct order because they use the oligonucleotides of the other strand as templates which cross the joins between adjacent oligonucleotides. An example of self-templating oligonucleotides is shown in FIG. 1.

When the set of oligonucleotides is annealed together, they template each other and assemble in the correct order to provide the whole double stranded polynucleotide sequence but with breaks in the phosphodiester backbone where the gaps between adjacent oligonucleotides are. These gaps are only the distance between adjacent base pairs so the 3′ end of one oligonucleotide is very close to the 5′end of the next adjacent oligonucleotide.

A covalent bond can be formed chemically between the adjacent 3′ and 5′ ends of adjacent oligonucleotides on the same strand to form a double stranded polynucleotide with no breaks in either of the strands.

The chemistry of the covalent bond joining the 3′ end of one oligonucleotide to the 5′ end of the next oligonucleotide may be chosen to form a covalent linkage that can be read through in vitro and in vivo by natural polymerases and transcription factors so that the resulting polypeptide can be transcribed and translated in vivo to express a protein encoded by the polynucleotide. The covalent linker may be a linker that can be repaired by the cellular DNA machinery to a phosphodiester bond prior to transcription.

The covalent bond joining the oligonucleotides may formed by a chemical process in vitro. The covalent bond joining the oligonucleotides may not be a natural phosphodiester bond. The covalent bond joining the oligonucleotides may not be made by enzymes. The covalent bond joining the oligonucleotides may not be made by a ligase.

In order to use a chemical method to create a covalent bond between the 3′ end of one oligonucleotide and the 5′ end of the adjacent oligonucleotide, the 3′ end and/or the 5′ end of one or more, or each, oligonucleotide may be chemically modified to introduce a functional group that is able to react to form a bond with the functional group on the adjacent oligonucleotide. The functional groups on all of the 3′ ends of all of the oligonucleotides may be the same functional group. The functional groups on all of the 5′ ends of all of the oligonucleotides may be the same functional group. The functional groups on 3′ ends of all of the oligonucleotides may be different from the functional groups on the 5′ ends of all of the nucleotides. All of the functional groups may be the same. The functional group on the 5′ end of one oligonucleotide may be specifically chosen to react with and form a covalent linkage with the functional group on the 3′ end of an adjacent oligonucleotide to form a linkage that can be read through by polymerases and transcription factors in vivo.

Functionalised means that a functional group has been added. Where the end of an oligonucleotide has been functionalised, a functional group has been added to the end of the oligonucleotide.

The functional groups may be any groups that react to form disulphide bonds, amides, alkenes, alkanes, or may be any two heteroatoms that can be joined together or any heteroatom joined to a carbon atom.

The chemical reaction between the functional group at the 3′ end of one oligonucleotide and the functional group at the 5′ end of the adjacent oligonucleotide may be spontaneous or initiated by addition of a catalyst or further chemical.

Where the functional groups are an alkyne and an azide the catalyst may be Cu (I) which catalyses the reaction to form a triazole phosphodiester mimic or no catalyst may be required.

The catalyst may not be an enzyme. For example, the catalyst may not be a ligase. The covalent linkage between the oligonucleotides is formed using a chemical process in vitro. This is advantageous because a chemical process has a higher yield and can be done on a larger scale. A chemical process does not depend on enzymes, which can contain impurities.

The reaction may occur upon the addition of additional reagents (e.g. coupling reagents) to initiate the reaction or occur due to the proximity of the functional groups once self-assembly of the gene has occurred.

When synthetic oligonucleotides are ligated together chemically any epigenetic markers or modified bases that are in the oligonucleotides are unchanged and this allows large open reading frames, whole genes or a whole genome to be synthesised chemically on a large scale including epigenetic markers.

The chemical linkages between the oligonucleotides are read through by enzymes in vivo. A polynucleotide or gene with more than 2, more than 3, more than 4, more than 5, more than 10 or more than 15, more than 30, more than 50 or more than 100 chemical linkages may be read through by RNA and/or DNA polymerases, prokaryotic and/or eukaryotic transcription factors and/or DNA replication machinery in vitro or in vivo.

The method may be a one-pot ligation. This means that the oligonucleotides are designed to template each other so that the oligonucleotides assemble in the correct orientation and the correct order when they are annealed together and the oligonucleotides can all be ligated together in one chemical reaction that forms bonds between each of the oligonucleotides in the same reaction to form a double stranded polynucleotide. the double stranded polynucleotide may encode a whole gene.

An advantage of the present method is that assembly of the oligonucleotides and joining of the oligonucleotides to form the final product can be carried out without the need for a solid support such as resin beads. Because the oligonulceotides are self-templating they can self-assemble in the correct order in solution without splints or additional oligonucleotides or solid supports. Therefore, an advantage of the present method is that it provides a one-pot method for assembling oligonucleotides to form a polynucleotide that can be a whole gene or genome in solution.

The double stranded polynucleotide may be at least 300 base pairs, at least 400 base pairs, at least 500 base pairs, at least 800 base pairs, at least 1000 base pairs, at least 1500 base pairs, at least 2000 or at least 5000 base pairs long.

Each of the oligonucleotides may be at least 30 base pairs, at least 50 base pairs, at least 70 base pairs, at least 100 base pairs or at least 200 base pairs long. Each oligonucleotide may be between 30 and 200 base pairs long.

One or more of the oligonucleotides may be chemically synthesised using standard chemical oligonucleotide synthesis methods known in the art. One or more of the oligonucleotides may be produced using any standard laboratory technique. The oligonucleotides may all be chemically synthesised. The oligonucleotides may be chemically synthesised with functional groups attached to the 5′ and/or 3′ ends.

The oligonucleotides may be synthesised including non-standard bases and/or epigenetic modifications, for example methylated bases. An advantage of the present method is that many copies of the polynucleotide can be made by chemical synthesis and chemical ligation and the epigenetic modifications remain unchanged. This is in contrast to amplification by PCR techniques, which do not recognise non-standard bases and epigenetic modifications such as methylation and do not replicate them.

The polynucleotide sequence, gene or genome may include epigenetically modified bases, including methyl-cytosine, hydroxymethyl-cytosine, formyl cytosine, methyl adenine, hydroxymethyladenine or other chemical moieties, such as a fluorescent group, or added chemical functional groups for further decoration of the gene after assembly (for example bromination).

One method of chemical ligation that can ligate oligonucleotides together by forming a link between the oligonucleotide backbones is a triazole phosphodiester mimic in RNA as described in El-Sagheer and Brown 2010, PNAS vol. 107 no. 35, 15329-15334 and is also a phosphodiester mimic in DNA that can be read through by DNA and RNA polymerases as described in El-Sagheer et al. 2011, PNAS vol. 108 no. 28, 11338-11343. Both of the above publications are incorporated herein in their entirety. The 5′ end of an oligonucleotide is functionalised with an alkyne group and the 3′ end of an adjacent oligonucleotide is functionalised with an azide group and the covalent bond formed between the two functionalised groups is a triazole phosphodiester mimic. The reaction is catalysed by Cu(I).

The reaction to link the oligonucleotides may form a triazole phosphodiester mimic and follow the reaction scheme below or an RNA equivalent thereof:

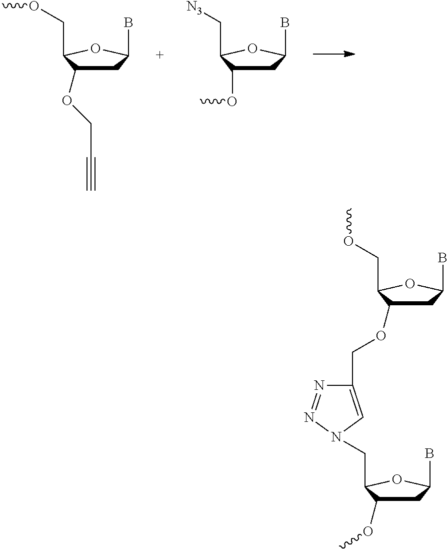

This reaction provides a triazole phosphodiester mimic that has an overall shape similar to that of a phosphodiester group.

The method of the present disclosure may further comprises the steps of:

I) Ligating the double stranded polynucleotide into a vector, preferably an expression vector; and

II) Transforming cells with the expression vector such that the cells express the open reading frame.

The polynucleotide may be a whole open reading frame or a whole gene and may include upstream and downstream regulatory elements. The polynucleotide may be ligated into a vector. The polynucleotide may be ligated into an expression vector that is suitable for expressing the protein encoded by the open reading frame in cells. The vector may be suitable for expressing the open reading frame in prokaryotic cells, for example bacterial cells, eukaryotic cells, such as animal cells and/or human cells.

The polynucleotide may be designed or arranged to complement the vector that it will be ligated into to ensure that, once the polypeptide is ligated into the vector all of the necessary sequences are present to allow the polypeptide to be expressed in the chosen cell type.

The polynucleotide may be directly transformed in to a cell (prokaryote or eukaryote) or may be designed with ends that are suitable for ligating into a vector. For example the polynucleotide may be made with overhangs at each end or with restriction sites at each end that can be cleaved to provide ends that are suitable for ligating into the chosen vector.

The vector comprising the polynucleotide may be transformed into any suitable type of cells. As the vector/polynucleotide construct may be constructed with all of the necessary sequences for expression the cells may express a protein encoded by the open reading frame.

In order to facilitate expression in prokaryotic or Eukaryotic cells, each terminus of the gene may be pre-designed to include sticky ends for direct ligation into an expression plasmid. The start of the assembled polynucleotide may contain transcription factor, or polymerase binding sites and/or non-natural nucleosides (such a locked nucleic acids) to prevent degradation of the linear gene product.

The polynucleotide may not be ligated into a vector.

The polynucleotide may be designed to comprise 2-3 or more locked nucleic acids at each end so that it can remain linear and be transformed into cells as a linear piece of DNA. Transforming linear DNA is particularly advantageous for eukaryotic cells, for example human cells.

In a second aspect the present disclosure provides a double stranded DNA sequence made by the method of the disclosure. The double stranded DNA sequence may comprise at least a complete open reading frame and may comprise regulatory sequences or a gene.

In a further aspect, the present disclosure provides an expression vector comprising a double stranded DNA sequence made by the method of the disclosure and comprising at least a complete open reading frame.

In a further aspect, the present disclosure provides a cell comprising a DNA sequence made by the disclosed methods and comprising a double stranded DNA sequence comprising at least a complete open reading frame.

Thus, in various embodiments, the present disclosure relates to the use of click-DNA ligation for the fully chemical, one-pot assembly of oligonucleotides into a gene. The potential of this method is demonstrated by synthesizing the 335 base-pair gene encoding the green fluorescent protein iLOV from ten functionalized oligonucleotides containing 5′-azide and 3′-alkyne units. The resulting click-linked iLOV contains eight triazoles at the sites of click-ligation in its backbone, yet is fully biocompatible. Click-linked iLOV is replicated by DNA polymerases in vitro and encodes a functional iLOV protein in E. coli. This fully chemical approach to gene synthesis may be employed for the construction of epigenetically modified genes and genomes.

In this strategy, a highly specific and selective chemical reaction is used to join oligonucleotides with the required functional groups at each terminus (FIG. 1A). Given the self-tem plating properties of DNA, a one-pot assembly strategy can be envisaged whereby the sense and antisense strands of the desired gene are segmented into overlapping fragments that are chemically synthesized as functionalized oligonucleotides (FIG. 1Ai). These are annealed in the correct order to bring the neighbouring functional groups in sufficient proximity to enable bond formation (FIG. 1Aii). The chemical reaction between the functionalized termini may be initiated through the addiction of a catalyst, leading to covalent bonding between the oligonucleotides to give the desired gene (FIG. 1Aiii). However, an absolute requirement for the utility of such a process is the tolerance of the chemical ‘scar’ formed at the site of ligation (in place of the phosphodiester bond) in biological systems, so that the resulting DNA strand is correctly replicated and transcribed in cells.

A possible chemical reaction for this purpose is the copper-catalyzed azide-alkyne cycloaddition (CuAAC), which has been used by us and others to link oligonucleotides functionalized with a 3′-propargyl and a 5′-methylene azide (FIG. 1B). The resulting click-linked DNA backbone has been shown to be accurately replicated and transcribed in bacterial and human cells when a single triazole linker was incorporated into each strand of a DNA duplex. In addition, the linker has been shown to be accurately transcribed in vitro by T7 RNA polymerase, while structural studies illustrated minor disturbance to the double helix structure from a single click-linker. The present inventors have surprisingly shown that it is possible to incorporate multiple non-natural linkers, in this case multiple click-linkers into the same DNA strand and that they are tolerated by living systems. This is surprising because the efficiency of natural polymerases was thought to decrease with just one non-natural linkage in an in vivo system. The present inventors have taken this a step further by combining the use of multiple non-natural linkers, for example multiple click-DNA ligation reactions, with the self-assembling properties of DNA for one pot gene synthesis that creates a gene that can be expressed with significant efficiency in vivo and can retain epigenetic information.

### DISCUSSION

All previous gene synthesis methods use enzymes to assemble synthetic oligonucleotides into gene- or genome-sized fragments. While these methods have been pushed to their limit, achieving challenging feats such as the synthesis of whole prokaryotic genomes and a eukaryotic chromosome, the reliance on enzymatic assembly limits scalability as well as the ability to encode epigenetic information (which cannot be read or transferred by the enzymes used, and/or is erased during assembly). Yet the ability to include epigenetic information in synthetic genes will be critical in meeting the challenge of the next phase of DNA synthesis, namely the goal of synthesizing a functional human genome. It may be reasonably argued that given the extensive level of cytosine methylation and hydroxymethylation in the human genome, and the critical role it plays in gene regulation, synthesizing the human genome will only be meaningful and biologically relevant if it also contains epigenetic information. To overcome these limitations and enable the synthesis of epigenetically modified genes and genomes, the present inventors have demonstrated a fully chemical approach to gene assembly, using chemically modified oligonucleotides that are covalently bound into genes by a suitable chemical reaction. The inventors have demonstrated this possibility using click-chemistry and the CuAAC reaction; but it should be noted that the principle of chemical DNA ligation is not limited to this reaction and can (and should) be applied to a variety of chemical reactions. The one key requirement however, is that the functional group produced on the DNA backbone by chemical ligation is biocompatible. The inventors chose the CuAAC reaction owing to its fast reaction rate, high yield, compatibility with aqueous media, because azides and unactivated alkynes are orthogonal to the functional groups present in oligonucleotides, and its biocompatibility. However, there are many other examples of DNA backbone mimics that are biocompatible and could be successfully used in place of to CuAAC reaction for chemical DNA ligation.

Combining conventional oligonucleotide synthesis with chemical DNA ligation as demonstrated in the present disclosure, not only allows the synthesis of genes on the μg-mg scale bearing site-specific modifications ranging from epigenetic bases to larger bulky groups such as fluorophores, but also enables the automation of gene assembly, a critical step for scaling-up production and reducing the time taken to make a gene or genome.

Here, the inventors demonstrate the one-pot synthesis of the 335 bp iLOV gene by click-DNA ligation of ten doubly-modified oligonucleotides. The resulting click-iLOV construct has eight triazole moieties in its backbone, yet is fully biocompatible in E. coli. The inventors isolated 95 μg of the click-linked iLOV gene after purification, a challenging feat when using conventional enzymatic methods. The inventors initially compared the properties of click-linked iLOV to the canonical equivalent (generated by PCR) in vitro; CD spectroscopy showed that both had similar secondary structures, and despite the presence of eight triazole backbone linkers, the melting temperature of click iLOV was only 3° C. lower than the canonical gene. They also observed that DNA polymerases can read through the click-linked iLOV.

The inventors next assessed the biocompatibility of the click-iLOV gene in E. coli. To distinguish mutations in the progeny of the click-iLOV gene caused by the triazole linkers from those arising from oligonucleotide synthesis, or cloning, the inventors assembled a control gene using T4 DNA ligase with ten oligonucleotides (canonical equivalents of those used for click-ligation). Interestingly, it was found that there are fewer mutations in iLOV genes isolated from cells transformed with click-linked iLOV than in those isolated from cells transformed with ligase-linked iLOV, both in terms of ratio of functional genes produced (58.3±11.2% for click-linked iLOV versus 39.5±13.6% for ligase-linked iLOV), and ratio of functional genes containing errors (2% for click-linked iLOV versus 16% for ligase-linked iLOV). The mutations observed in the click-linked gene were not located at, or adjacent to, the sites of click-ligation, and are therefore unlikely to be a consequence of the triazole-linked backbone. Given the similarity in errors between the click-linked and canonical control gene, the most likely cause of these errors lies in the oligonucleotide synthesis and purification steps, and this may account for the higher error rates when using non-modified oligonucleotides. As they observed an effect from the multiple triazole linkers on PCR amplification in vitro, they next assessed the contribution of NER to the observed biocompatibility in cells. The inventors used a UvrB-deficient E. coli strain (incapable of NER) and observed a similar rate of error in the iLOV sequence isolated from the progeny of cells transformed with click-linked iLOV as for those transformed with ligase-linked iLOV. These data indicate that the triazole-linkers are truly biocompatible and not repaired. In this respect, had repair-mediated conversion of the triazole linkers to phosphodiester linkers been the origin of the observed biocompatibility, this would not necessarily have been a problem, as the sequence of the opposing strand (to the click-linker) used as a repair template is canonical and contains the correct genetic information. Regardless, the experiments demonstrate the viability of using a chemical ligation strategy for gene synthesis.

Gene synthesis has been driven forward by new techniques and technologies. The one-pot click-mediated DNA ligation approach presented here offers an alternative, fully chemical approach to gene assembly and has the potential to respond to the challenge of synthesizing epigenetically modified genes and genomes.

FIG. 1 shows how gene assembly via this one-pot click-ligation strategy and demonstrate the biocompatibility of the resulting triazole-containing DNA in E. coli. The viability of this approach is illustrated with the assembly of the 335 bp gene that encodes the fluorescent protein iLOV from ten functionalized oligonucleotides (FIG. 1C). This work serves as proof-of-concept for enzyme-free gene assembly and paves the way for the fully chemical synthesis of genes and genomes.

Results

Synthesis of Oligonucleotides Comprising the iLOV Gene.

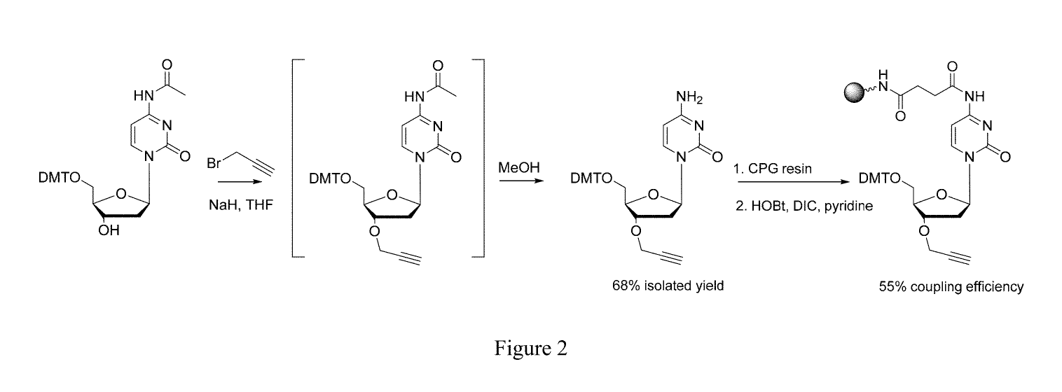

The 335 bp gene encoding iLOV was codon-optimized for expression in E. coli. The sites of ligation were between adjacent deoxycytidine (dC) and deoxythymidine (dT) residues (CpT steps), and positioned throughout the gene to give an overlap of at least 10 base pairs between the sense and antisense strands (FIG. 1C). Efforts were also made to keep the length of each synthetic oligonucleotide between 50-80 bases to enable high-fidelity synthesis and facilitate purification. The sequence of the oligonucleotides were not optimized to give favourable melting temperatures or avoid secondary structures; in fact the melting temperatures of the individual fragments differed by as much as 6.6° C. due to varying GC-contents. These oligonucleotides were synthesized by phosphoramidite chemistry using a resin functionalized with 3′-propargyl dC (FIG. 2). The monomer was isolated from a one-pot reaction in which N⁴-acetyl-2′-deoxy-5-O-DMT cytidine was alkylated using propargyl bromide in the presence of NaH in THF. Sonication for 2 h at room temperature drove the reaction to completion without the formation of any by-products. Methanol was subsequently added to quench the residual propargyl bromide and simultaneously remove the acetyl protecting group to give the monomer in 68% yield; an improvement on the previously published 2-step reaction to synthesize 3′-propargyl methyl-dC monomer. The monomer was then loaded onto an amino functionalized resin in preparation for DNA synthesis using a previously reported protocol. For oligonucleotides that possessed a 5′-azide, 5′-Iodo-2′,5′-dideoxythymidine-3′-phosphoramidite was incorporated as the final monomer during oligonucleotide synthesis. The azide functional group was installed by treating the resin bound oligonucleotide with sodium azide to displace the iodine as previously reported. The terminal fragments of the sense and antisense strands were synthesized to contain 5′-phosphate groups rather than azides. To enable ligation of the final synthetic gene into a plasmid, the sequences of the four terminal fragments were modified to include overhangs compatible with digestion with NdeI and EcoRI restriction endonucleases (FIG. 1C).

The purity of the oligonucleotides and the integrity of the azide and alkyne functional groups were crucial factors in ensuring successful assembly of the full length gene, and expected function of the protein product. Two different methods of purifying the oligonucleotide were therefore evaluated. The crude solutions of each oligonucleotide were divided into two fractions; one fraction purified by semi-preparative HPLC using a hexylammonium acetate/acetonitrile buffer system and the other via polyacrylamide gel electrophoresis (PAGE). The purity of the oligonucleotides was then quantified via analytical HPLC and capillary electrophoresis. It was observed that HPLC purification yielded purer oligonucleotides than PAGE purification. Furthermore, the inventors found that capillary electrophoresis tended to overestimate the purity of the oligonucleotides compared to analytical HPLC. Consequently, only the HPLC-purified oligonucleotides were used to assemble the iLOV gene.

Click-Mediated Assembly of Gene Encoding iLOV.

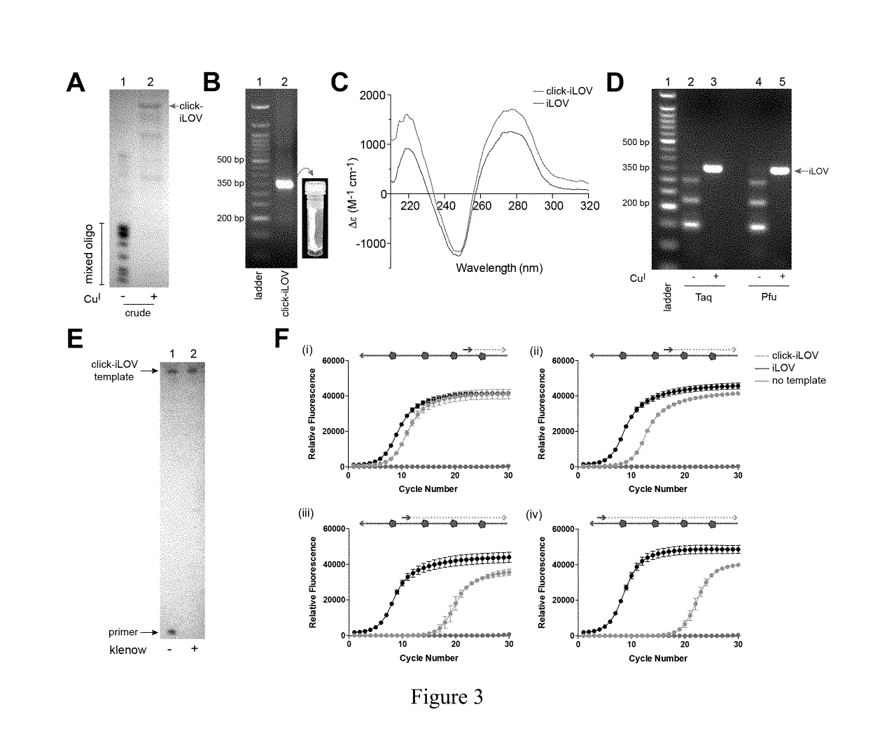

The ten oligonucleotide fragments synthesized above were combined in ascorbate salt solution, heated at 95° C. for 3 min, then cooled to room temperature over 2 h to enable annealing. The inventors hypothesized that the self-templating properties of DNA would cause the oligonucleotides to anneal to give the unligated iLOV gene. The alkyne- and azide-functionalized termini of these thermally assembled oligonucleotides were simultaneously reacted to form triazoles via addition of copper sulphate. A control assembly reaction containing modified oligonucleotides, but no copper was also carried out to assess the importance of click-linking the annealed DNA. The crude click reactions were purified by PAGE under denaturing conditions to ensure that any unreacted fragments or by-products migrated separately from the assembled gene. As expected, in the absence of the copper catalyst, the individual oligonucleotide fragments did not assemble to form the iLOV gene, but rather migrated individually towards the bottom of the gel (FIG. 3A, lane 1). In the copper-containing reaction however, a distinct high molecular weight band was observed (FIG. 3A, lane 2), indicating correct assembly and click-DNA ligation of the functionalized oligonucleotides. The lower molecular weight bands observed in the same lane were attributed to truncation or intramolecular click reactions, leading to cyclized oligonucleotide products. A residual unreacted oligonucleotide was also detected. Performing small-scale template mediated click-ligations identified this oligonucleotide as the F2 fragment and indicates a slight excess of this oligonucleotide in the reaction mixture. The band corresponding to the assembled iLOV gene was excised from the gel and the DNA was extracted to give 95 μg of pure, click-linked iLOV gene as a single band at ˜350 bp (FIG. 3B lane 2 and insert in FIG. 3B).

The Effect of Multiple Click-Linkers on the Secondary Structure of DNA.

A single triazole incorporated into the backbone of DNA has been shown to cause small distortions that result in displacement of the deoxyribose sugar and an increase in the distance between the bases flanking the triazole. Given this, the inventors were concerned that the presence of multiple triazoles might drastically perturb the secondary structure of the click-linked gene and affect its biocompatibility. The inventors therefore conducted circular-dichroism (CD) spectroscopy analysis to probe this. CD analysis of the click-linked iLOV gene gave bisignate signals with maxima at λ=+276/−248 nm which are characteristic of the B-type DNA helix, identical to that observed for the canonical iLOV gene (FIG. 3B). The lack of perturbation in the CD signal for click-linked iLOV when compared to the canonical equivalent demonstrates that the multiple backbone triazoles do not significantly alter the conformation of the DNA double helix. However, melting temperature analysis of the two constructs suggests that the triazoles do have a minor destabilizing effect. The canonical iLOV gave a melting temperature of 84.3° C. while that of the click-linked gene was 81.3° C., indicating that the eight triazoles destabilize the duplex by 3° C.

Replication of click-linked iLOV in vitro. The ability of DNA polymerases to replicate click-linked iLOV in vitro was next assessed. The inventors used click-linked iLOV as template for PCR amplification by either Taq or Pfu polymerase, and the unligated, modified oligonucleotides were used as a negative control. Click-linked iLOV was amplified by both DNA polymerases, giving a single band that appeared at ˜350 bp markers in the ladder (FIG. 3D, lanes 3 and 5). In contrast, PCR of the unligated oligonucleotides did not give rise to a band corresponding to the full length iLOV product. Instead a strong band was observed at ˜150 bp, with weaker bands visible at ˜275 and ˜300 bp (FIG. 3D, lanes 2 and 4). The absence of truncation products when amplifying the click-linked iLOV, suggests that the triazole backbone linkers do not cause the polymerase to detach from the template or stall at the triazole. The inventors also assessed the ability of DNA polymerases to read through click-linked iLOV using a primer extension assay, whereby a single strand of the click-linked DNA is copied in linear fashion by the Klenow fragment (exo)⁻ of DNA polymerase I (Klenow). This assay uses the sense strand of click-linked iLOV as template, with the polymerase processing through this strand every time. The single-strand, click-linked template required for the primer extension assay was generated using a template-mediated click-ligation strategy using the modified oligonucleotide fragments that comprised the sense strand of iLOV.

These oligonucleotides were assembled in the correct order using four shorter complementary splints overlapping the terminal regions of each modified oligonucleotide by 20-25 bases either side of the ligation points. The assembled sense strand was observed as a high molecular weight band at the top of the gel; as with the one-pot gene assembly, cyclization and truncation products were observed in addition to the residual unreacted oligonucleotides. The click-linked sense strand of iLOV was extended successfully by Klenow, depleting all the primer added to the reaction, with only a single band corresponding to the full length iLOV gene being observed (FIG. 3E, lane 2). The absence of any truncated species or unreacted primer indicates that Klenow successfully reads though the four triazole backbone-linkers, extending the primer to the full length complementary strand. As expected, the primer remained present in the negative control reaction lacking Klenow (FIG. 3E, lane 1).

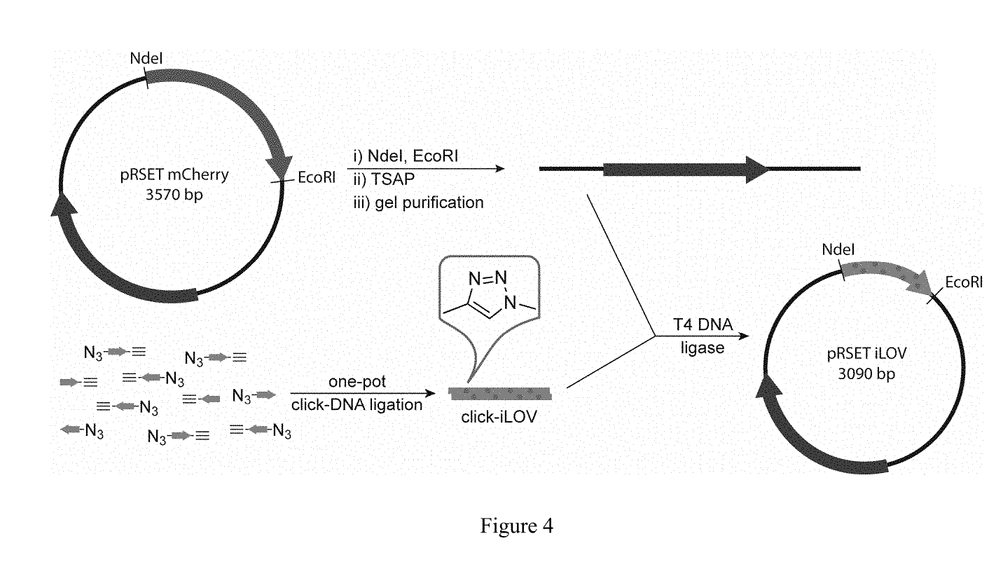

The inventors further probed the effect of DNA backbone triazoles on DNA polymerases using real-time PCR (qPCR). The inventors designed four primers to bind upstream of each triazole on the sense strand of click-linked iLOV, and monitored the rate of replication through the increasing number of triazoles by Taq polymerase. For comparison, the experiment was repeated using canonical iLOV as template. The inventors observed an inverse relationship between the number of triazoles in the DNA backbone and rate of PCR product formation (FIG. 3F). The inventors compared the threshold cycle numbers (Ct value) of the templates (the point at which the fluorescence is first detected as statistically significant above the threshold) for click-linked iLOV and the canonical equivalent. When reading through one triazole linker, the Ct values for the click-linked iLOV and canonical iLOV were comparable at 9.4±0.1 and 7.5±0.1 respectively (FIG. 3Fi). The difference in Ct values increased when amplifying through two triazoles to 11.3±0.1 for click-linked iLOV and 7.0±0.1 for canonical iLOV (FIG. 3Fii). When reading through three triazoles, the difference in Ct values further increased to 18.5±0.8 for click-linked iLOV and 6.6±0.2 for canonical iLOV (FIG. 3Fiii), while reading through four triazoles gave Ct values of 20.7±0.5 for click-linked iLOV and 6.3±0.1 for canonical iLOV (FIG. 3Fiv). This data suggested that increasing the number of triazoles in the backbone of the template DNA slows down DNA replication by Taq polymerase. It should be noted that although the number cycles required to detect a significant amount of PCR product above the baseline fluorescence increases with more triazoles, the flat line at zero during these early cycles (e.g. in FIG. 3Fiv) does not equate to the lack of DNA replication. Rather, the amount of DNA produced in the early cycle is below the minimum fluorescence threshold and so is reported as zero by the instrument. Furthermore, total product levels for the PCR reactions with click-linked iLOV approach that of the canonical equivalent by the end of the PCR reaction in all cases. To further probe this effect, the inventors repeated each amplification reaction (through increasing number of triazoles) with Taq DNA polymerase, and visualized the DNA produced on an agarose gel (Supplementary FIG. 4) and quantified the concentration of DNA produced (Supplementary Table 4). Interestingly, there no significant difference was observed between using click-linked iLOV or the canonical iLOV gene as template, even when reading through 4 triazoles. This suggests that the difference in replication rates observed by qPCR is normalized by the end of the PCR reaction (due to multiple cycles). However, it should be noted that any effect from the triazole-linkages will only be relevant during the first few PCR cycles; after this, there will be a large excess of unmodified template product, so amplification will proceed at the same rate in all reactions. It is unclear whether the observed triazole-dependent slowdown in PCR replication would also affect replication of click-linked iLOV in cells.

Probing the Biocompatibility of the Click-Linked iLOV Gene in E. coli.

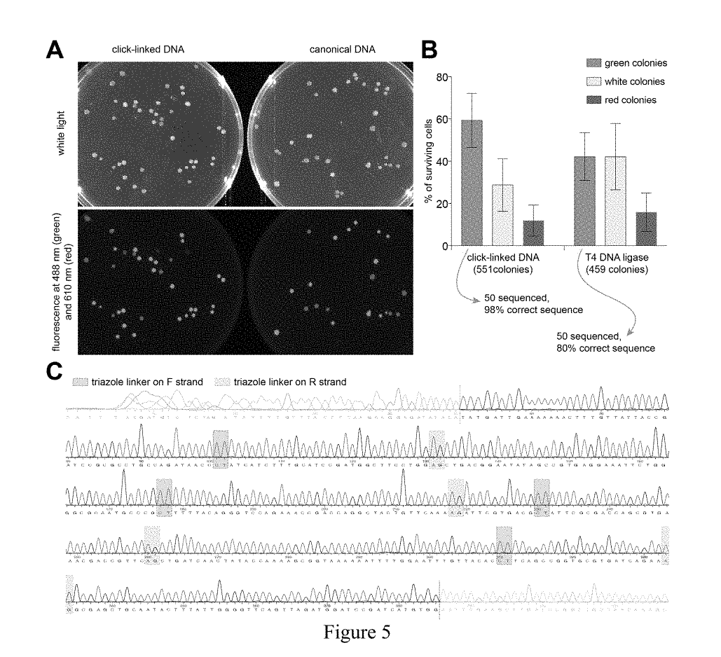

The biocompatibility of our clicked-linked gene was next probed in E. coli. For comparison, the inventors assembled the canonical iLOV gene with T4-DNA ligase, using 5′-phosphorylated equivalents of the oligonucleotides used for click-ligation. Both the click-linked iLOV and ligase-assembled iLOV were designed to contain sticky ends for ligation into the pRSET-mCherry plasmid²⁴ cleaved by NdeI and EcoRI restriction endonucleases (FIG. 4). These restriction sites were chosen so that the click-linked gene, which encodes the green fluorescent protein iLOV, directly replaces the gene encoding for the red fluorescent protein mCherry in the backbone. Thus surviving colonies containing click-linked iLOV would be readily distinguished from false positives containing the parent plasmid by the colour of their fluorescence. The pRSET-mCherry plasmid was sequentially digested with NdeI and EcoRI restriction endonucleases, which removes the gene encoding mCherry, and treated with shrimp alkaline phosphatase to remove the terminal phosphate groups to minimize backbone-only ligation. Click-linked iLOV was ligated into the purified digested vector with T4 DNA ligase. The ligation mixture was transformed into KRX E. coli cells, chosen as they contain a chromosomal copy of T7 RNA polymerase and therefore allow rhamnose-dependent overexpression of iLOV from the click-linked gene. Transformed cells were plated on LB agar, incubated overnight and the resulting colonies were assessed for the green fluorescent phenotype associated with transcription and translation of click-linked iLOV (FIG. 5A). Of the 551 assessed colonies (n=10) 58.3±11.2% displayed the green fluorescent phenotype associated with iLOV, while 28.4±10.1% were white and 13.3±10.5% were red (FIG. 5B). When using the ligase-assembled iLOV, of the 459 assessed colonies (n=10) 39.5±13.6% were green, 43.9±11.1% were white and 16.6±14.5% were red (FIG. 5B). It is interesting to note that in both cases ˜15% of the colonies still contained undigested or singly digested and religated pRSET-mCherry, suggesting poor performance by one of the restriction endonucleases despite gel purification of the double digested backbone and treatment with shrimp alkaline phosphatase (to prevent backbone-only ligation). There are two possibilities that would lead to white colonies; either the gene for iLOV in those colonies is mutated, or the plasmid does not contain an insert. To further probe this, the inventors isolated and sequenced the plasmids from 25 white colonies; 92% of these colonies contained point mutations that led to frameshifts or deletions in the iLOV gene, while the remaining 8% of colonies contained religated vector without an insert. Plasmids from 50 colonies expressing the green phenotype of the click-linked iLOV gene were isolated and their sequences were analyzed. Of these colonies, 49 (98%) possessed the correct sequence, meaning that the whole iLOV gene, including the regions flanking the triazole linkers, is correctly replicated in E. coli (FIG. 5C). The lone mutant contained an adenine to cytosine point mutation at thirteen bases after the second triazole (position 157 on the sense strand) which did not affect iLOV function. In comparison, of the 50 green colonies sequenced from the cohort transformed with the ligase-assembled iLOV gene, 40 (80%) were error-free while the remaining 10 (20%) contained point mutations at a variety of different positions. It is interesting that the error rate is higher for ligase-assembly than click DNA ligation, both in terms of number of colonies displaying green fluorescence (43.9±11.1% for ligase versus 58.3±11.2% for click) and mutations in the iLOV gene from green colonies (16% for ligase versus 2% for click). However, this may be a reflection of errors incurred during oligonucleotide synthesis, or more stringent purification of the modified oligonucleotides, rather than a consequence of the chosen method of assembly.

Assessing the Role of DNA Repair in the Biocompatibility of Click-Linked iLOV.

The green fluorescent phenotype observed in the majority of colonies transformed with click-linked iLOV and subsequent sequencing analysis confirmed the biocompatibility and high fidelity replication of this click-linked gene in E. coli (FIGS. 5A and 5B). However, it is possible that the triazoles present on the DNA backbone of click-linked iLOV are being converted to the canonical phosphodiester linkage via activation of the nucleotide excision repair (NER) system rather than being directly replicated in E. coli. To probe this possibility, a doubly-digested pRSET plasmid ligated with click-linked iLOV as insert (as above) was transformed into a AUvrB strain of E. coli (JW0762-2); UvrB protein is an essential component of the DNA damage recognition complex which is responsible for excising DNA lesions in the NER system. If biocompatibility of click-linked iLOV was due to excision of the triazole-linkers through DNA repair, then this would be lost in the AUvrB strain of E. coli, which is incapable of NER. As JW0762-2 does not contain a copy of T7 RNA polymerase, iLOV will not be transcribed from the pRSET plasmid, therefore the green fluorescent phenotype cannot be used as an indication of biocompatibility. The inventors therefore isolated and sequenced plasmids from 50 colonies transformed with click-linked iLOV and 50 colonies transformed with ligase-linked iLOV. Of the 50 copied of click-iLOV sequenced, 30 plasmids (60%) were error-free while the remaining 20 plasmids (40%) contained mutations; either simple point mutations (36%) or missing two or more consecutive bases (4%). Similar results were obtained for ligase-linked iLOV; 30 plasmids (60%) were error free while the remaining 20 (40%) contained mutations; either simple point mutations (28%) or missing two or more consecutive bases (12%). The high rate of error observed is likely a consequence of a lack of NER in these cells. Nonetheless, the similarity in the ratio of error-free clones observed when using click-linked iLOV or ligase-linked iLOV suggests that the cellular machinery of E. coli is able to correctly read through the multiple triazoles contained in click-linked iLOV.

Methods

For complete experimental methods see Supplementary Information.

One-Pot Assembly of iLOV Gene.

The oligonucleotides which comprised the sense and antisense strands of iLOV (F1-F5 and R1-R5, 4 nmol each, were combined and lyophilised. The oligonucleotides were resuspended in 0.2 M NaCl (400 μL) then annealed by heating at 95° C. for 15 min then gradually cooled to room temperature over 2 h; the temperature was reduced by 10° C. every 15 min. A Cu′ click catalyst solution was prepared from tris(3-hydroxypropyltriazolylmethyl)amine (0.7 μmol in 0.2 M NaCl, 154 μL), sodium ascorbate (1.0 μmol in 0.2 M NaCl, 14 μL) and CuSO₄.5H₂O (0.1 μmol in 0.2 M NaCl, 7 μL). The pre-mixed catalyst solution (160 μL) was added and mixture thoroughly degassed using argon then left at room temperature for 2 h. Formamide (560 μL) was added and the samples analyzed by denaturing 8% polyacrylamide gel electrophoresis by applying 550 V for 3.5 h. Bands corresponding to the assembled gene were excised and extracted from the gel using the ‘crush and soak method’. In brief, the excised polyacrylamide pieces were broken down into small pieces then suspended in distilled water (25 mL). The suspension was shaken at 37° C. for 18 h then filtered through a plug of cotton wool. The filtrate was concentrated to approximately 2 mL then desalted using through two NAP-25 and one NAP-10 columns. The desalted eluent was lyophilised prior to use.

Ligation of Click-Assembled iLOV Gene into pRSET Backbone.

The pRSET backbone required for ligation was prepared from the double digestion of pRSET mCherry. The plasmid was digested sequentially between the NdeI and EcoRI restriction sites using enzymes and buffer supplied by New England Biolabs, UK. Restriction digestions were performed in a 50 μL reaction volume with between 1-2.5 μg of plasmid, 10× CutSmart® buffer (5 μL) and the restriction enzyme (20 U/μg plasmid). The restriction digestion reactions were incubated at 37° C. (60 min/μg plasmid) then the 5′-terminus dephosphorylated by addition of shrimp alkaline phosphatase (1 U/μg plasmid, New England Biolab, UK). The reactions were incubated at 37° C. for a further 30 min then the enzymes inactivated by incubating at 70° C. for 10 min. The digested plasmid was analyzed by gel electrophoresis using 1% agarose gel in 1× Tris/Borate/EDTA buffer (TBE) by applying 100 V for 30 min. The band corresponding to the backbone excised and the DNA was isolated using GeneJet PCR Purification Kit (Thermo Fisher Scientific, UK). Ligation of click-ligated iLOV gene was performed in a total volume of 10 μL using 50 ng of pRSET vector, T4 DNA Ligase (1 μL, 3 U, Promega UK) and T4 DNA Ligase 10× reaction buffer (1 μL, Promega). The lyophilised click-ligated gene was resuspended in ultrapure water and an aliquot diluted to a concentration of 19.7 ng/μL. The click-ligated gene was added to the ligation reaction to give molar ratios of 1:1, 1:3 and 1:5 backbone:insert. Negative control ligations were set up as above, using water instead of insert. The ligation reactions were incubated at 4° C. for 16 h then at room temperature for 1 h. The T4 DNA ligase enzyme was subsequently deactivated by heating at 70° C. for 10 min.

Transformation of pRSET-iLOV into E. coli.

The inactivated ligation reactions were dialysed for 1.5 h against ultrapure water using a 0.025 μm membrane filter (Millipore, Cat No: VSWP02500). The recovered ligation mixtures (approximately 7 μL) were added to frozen aliquots of electrocompetent KRX cells (100 μL). Electroporation of the plasmids was achieved using MicroPulser system (BioRad) and standard protocols. The transformants were immediately recovered using ice-cold SOC medium (890 μL) then incubated at 37° C. for 1 h. An aliquot (100 μL) of the recovered cells were spread on LB agar plates supplemented with carbenicillin (100 μg/μL) and rhamnose (0.1% w/v) then incubated at 37° C. for 18 h. Individual colonies were selected and grown in LB Broth (25 mL) supplemented with carbenicillin (100 μg/μL) at 37° C. for 16 h. Plasmid DNA was extracted from these cells using a QiaPrep® Miniprep kit. The plasmids were sequenced by Eurofins MWG Operon (Ebersberg, Germany) using the T7 forward and reverse primers. Trace files were aligned against reference sequences using Clustal Omega web-based software.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of synthesising a double stranded polynucleotide comprising:
providing a set of self-templating oligonucleotides wherein the set of self-templating oligonucleotides encodes a double stranded polynucleotide sequence of interest;
annealing the oligonucleotides together so that they form the double stranded polynucleotide sequence with the oligonucleotides in the correct order and adjacent oligonucleotides in the same strand close enough to each other that a covalent bond could form between the 5′ end of one oligonucleotide and the 3′ end of an adjacent oligonucleotide; and
covalently bonding the backbones of adjacent oligonucleotides in the same strand to each other so that a covalent bond is formed between the 5′end of an oligonucleotide to the 3′end of the adjacent oligonucleotide to provide the double stranded polynucleotide of interest, wherein the covalent bonds between adjacent oligonucleotides can be read-through accurately by a DNA polymerase or an RNA polymerase.

2. The method according to claim 1, wherein the 5′ and/or 3′ ends of adjacent oligonucleotides are functionalised to enable a specific and selective chemical reaction to form a covalent bond between a functionalised group on the 5′ end of an oligonucleotide and a functionalised group on the 3′ end of an adjacent oligonucleotide.

3. The method according to claim 1, wherein the 5′ and/or 3′ ends of adjacent oligonucleotides are functionalised to enable formation of disulphide bonds, amide bonds, alkene bonds, alkane bonds, bonds between any two heteroatoms joined together or bonds between any heteroatom joined to carbon.

4. The method according to claim 1, wherein the set of oligonucleotides comprises at least two oligonucleotides encoding the sense strand of the DNA and at least two oligonucleotides encoding the antisense strand of the DNA.

5. The method according to claim 1, wherein the set of oligonucleotides covers the whole sequence of the double stranded DNA.

6. The method according to claim 1, wherein the chemical reaction between the functionalized termini is initiated by addition of a catalyst.

7. The method according to claim 1, wherein the catalyst is not an enzyme, for example, not a ligase.

8. The method according to claim 1, wherein the catalyst is Cu (I).

9. The method according to claim 1, wherein the covalent bonds between each oligonucleotide can be read through by DNA and/or RNA polymerase enzymes, Prokaryotic and/or Eukaryotic transcription factors, and DNA and/or RNA replication machinery in vivo.

10. The method according to claim 1, wherein all of the oligonucleotides in the set are annealed together in a single reaction.

11. The method according to claim 1, wherein the double stranded DNA comprises a gene.

12. The method according to claim 1, wherein the double stranded DNA is at least 300 base pairs in length.

13. The method according to claim 1, wherein each of the oligonucleotides is at least 70 base pairs in length.

14. The method according to claim 1, wherein at least one of the oligonucleotides in the set of oligonucleotides is a chemically synthesised oligonucleotide.

15. The method according to claim 1, wherein all of the oligonucleotides in the set are chemically synthesised oligonucleotides.

16. The method according to claim 1, wherein at least one of the oligonucleotides comprises at least one epigenetic modification.

17. The method according to claim 1, wherein at least one of the oligonucleotides comprises at least one methylated base.

18. The method according to claim 1, wherein at least one of the oligonucleotides comprises at least one modified or non-natural nucleotide.

19. The method according to claim 1, wherein the oligonucleotides are covalently joined together without using a ligase.

20. The method according to claim 1, wherein the 5′ end of an oligonucleotide is functionalised with an alkyne group, the 3′ end of an adjacent oligonucleotide is functionalised with an azide group, and the covalent bond formed between the two functionalised groups is a triazole phosphodiester mimic.

21. The method according to claim 1, wherein the method further comprises the steps of:
i) ligating the double stranded polynucleotide into a vector, preferably an expression vector; and
ii) transforming cells with the expression vector such that the cells express the open reading frame.

22. A double stranded DNA sequence made by the method of claim 1.

23. An expression vector comprising a DNA sequence made by the method of claim 1.

24. A cell comprising a DNA sequence made by the method of claim 1.

25. A cell comprising an expression vector comprising a sequence made by the method of claim 1.


SEQUENCE LISTING (1 sequence)

SEQ ID NO: 1 - DNA, 341 bp, Artificial Sequence
  Synthetically produced iLOV gene
catatgattgaaaaaaactttgttattaccgatccgcgcctgccagataaccctatcatctttgcatccgatggcttcctggagctgacggaatatagccgtgaggaaattctggggcgcaatgcccgctttttacagggtccagaaaccgaccaggctactgttcaaaagattcgtgacgctattcgcgaccagcgtgaaacgaccgttcagctgatcaactataccaaaagcggtaaaaaattttgaatttgttacaccttcagccggtgcgtgatcagaaaggcgagctgcaatactttattggggttcagttagatggatccgatcatgtggaattc